(19) United States
(12) Patent Application Publication
(10) Pub. No.: US 2007/0067032 A1
(43) Pub. Date: Mar. 22, 2007

(54) Meniscus preserving implant method and apparatus

(76) Inventors: Jeffrey C. Felt, Greenwood, MN (US); David Griffin, Vero Beach, FL (US); Mark A. Rydell, Golden Valley, MN (US); Paul J. Buscemi, Long Lake, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/562,781

(22) PCT Filed: Jun. 25, 2004
(86) PCT No.: PCT/US04/20458

§ 371 Date: Aug. 22, 2006

Related U.S. Application Data
(60) Provisional application No. 60/483,500, filed on Jun. 27, 2003.

(51) Int. Cl. A61F 2/38; A61F 2/30; A61F 2/46

(52) U.S. Cl. 623/14.12; 623/20.32

(57) ABSTRACT

The invention includes an interpositional arthrosplasty implant adapted to be retained in position in apposition to ajoint surface, at least in part, by surrounding healthy tissue. In some embodiments the implant comprises a knee implant. The implant can include one or more structural features adapted to be fixedly positioned within and/or in apposition to the natural meniscus.

### TECHNICAL FIELD

In one aspect, this invention relates to biomaterials for implantation and use within the body. In yet another aspect, this invention further relates to the field of orthopedic implants and prostheses, and more particularly, for implantable materials for use in orthopedic joints.

### BACKGROUND OF THE INVENTION

Applicant has previously described, inter alia, prosthetic implants formed of biomaterials that can be delivered and finally cured in situ, and/or that can be partially or fully prepared ex vivo, for implantation into the body, e.g., using minimally invasive techniques. See for instance, U.S. Pat. Nos. 5,556,429; 5,795,353; 6,140,452; 6,306,177; and 6,652,587, as well as US Application Publication Nos. US-2002-0156531; US-2002-0127264; US-2002-0183850; and US-2004-0107000, and International applications having Publication Nos. WO 95/30388; WO 98/20939; WO 02/17821; WO 03/053278; WO 03/061522, and WO 2004/006811 (the disclosures of each of which are incorporated herein by reference).

In spite of developments to date, there remains a need for a joint prosthesis system that provides an optimal combination of properties such as ease of preparation and use, and performance within the body, and particularly for use in those patients in which some meniscal tissue remains healthy, and might be retained.

### SUMMARY OF THE INVENTION

The present invention relates to orthopedic implants and prostheses adapted to be positioned within (e.g., inserted into) orthopedic joints, in order to provide a weight bearing, articulating, or other mechanical and/or structural feature or function. An exemplary embodiment of the present invention provides an implant suitable for insertion into a joint selected from the group consisting of those that provide immovable articulations (synarthroidal), mixed articulations (amphiarthroidal, e.g., the lumbar joint of the back), and movable articulations (diarthroidal, such as the knee, including both the medial and/or lateral compartments). The ability of amphiarthroidal and diarthroidal joints to provide effective and pain-free articulation, and/or to serve their weight-bearing function, is generally dependent on the presence of intact, healthy fibrocartilage and/or hyalin cartilage within the joint.

The present invention provides an interpositional arthrosplasty implant adapted to be retained in position in apposition to a joint surface, at least in part, by surrounding healthy tissue, such as the meniscus of the knee. This can be compared, for instance, to implants that are either free moving within or upon a joint, or that are substantially retained upon a joint surface by other means, such as by the use of anchors or sutures, by physical conformation and congruence with the supporting joint surface, and/or by contact with the opposing joint surface.

An implant of this invention can be comprised of biomaterials and can be used and prepared by means of corresponding methods and systems described herein. In turn, the method and system, including interpositional arthroplasty implant, are particularly useful for those patients that retain some healthy meniscal tissue. In such patients, the implant of this invention can provide the various benefits, including an improved combination of comfort, alignment, cushioning, and long term performance.

In one preferred embodiment, the implant includes one or more structural features adapted to be fixedly positioned within and/or in apposition to the healthy meniscus, in a manner that permits the implant to be retained by the meniscus in a manner that further improves the retention of the implant itself upon the tibial surface.

In some embodiments, the present invention provides an interpositional arthroplasty implant for insertion into the lateral and/or medial cavity of a knee joint. The implant has a first major surface and a second major surface. The first major surface is adapted to mate with the tibial plateau of a tibia, preferably in a substantially congruent and fixed relationship. In some embodiments, the first major surface is textured promote a fixed relationship with the tibia. The second major surface is adapted to be positioned as an articulating surface against a medial or lateral condyle of a femur. The second major surface is preferably dimensioned so as to provide a femoral glide path. Accordingly, in some embodiments the first major surface includes a generally centrally located depression. In some embodiments, the glide path is smooth to promote articulation. The implant can also include a relief extending below the second major surface. In some embodiments of the present invention, the relief defines a cavity having generally annular shape. In some cases, the cavity can be continuous or discontinuous, extending along either all or one or more portions of the circumference of the implant. In particularly advantageous embodiments of the present invention, the cavity is dimensioned and positioned to receive and/or itself be retained within some or all of the patient's own meniscal tissue.

The implant of the present invention is particularly useful for those patients that retain some healthy meniscal tissue, in that it can provide the various benefits associated with the use of an implant of the type described herein, e.g., restore alignment and provide an elastomeric cushioning, function, while preserving and benefiting from the presence of healthy tissue. Further, the implant of the present invention is particularly suited for use within the lateral compartment of a knee because the engagement of the implant with the meniscal tissue helps to stabilize the implant against forces that tend to be more prevalent in the lateral compartment.

Some embodiments of the system can also include one or more devices in the form of a kit that can be used to provide or perform some or all of the steps of preparing the joint to receive an implant, determining an appropriate implant size for a particular joint, determinig an appropriate implant thickness, inserting the implant into the joint, and/or securing the implant to a desired extent. One or more of the various components and devices, including optionally one or more implants themselves, can be provided or packaged separately or in varying desired combinations and subcombinations to provide a kit of this invention.

### BRIEF DESCRIPTION OF THE DRAWINGS

FIG. 1 is a partial medial side view of a right leg including an implant in accordance with an embodiment of the present invention.

FIG. 2(a) is a top view of an implant in accordance with an embodiment of the present invention.

FIG. 2(b) is a sectional view along Section B-B of the implant of FIG. 2(a).

FIG. 2(c) is a sectional view along Section C-C of the implant of FIG. 2(a).

FIG. 3 is a perspective view of a leg including an implant in the lateral compartment in accordance with an embodiment of the present invention.

FIG. 4 is a plan view of various components of a kit in accordance with an embodiment of the present invention.

FIG. 5 includes various views of a tibial smoother in accordance with an embodiment of the present invention.

FIG. 6 is a perspective view of a femoral smoother in accordance with an embodiment of the present invention.

FIG. 7 is a side plan view of a femoral smoother in an exemplary smoothing step of an embodiment of the present invention.

FIG. 8 is a side plan view of a implant template in an exemplary sizing step of an embodiment of the present invention.

FIG. 9 is a side plan view of a gripper in an exemplary insertion step of an enbodiment of the present invention.

FIG. 10 is a front view of a leg including an implant in the lateral compartment in accordance with an embodiment of the present invention.

FIG. 11 is a side view of a rasp in accordance with an embodiment of the present invention.

### DETAILED DESCRIPTION

In one preferred embodiment, the method and system involve the preparation and use of one or more components (e.g., polymeric, ceramic, and/or metallic) for insertion and placement into the body. The method and system permit the on site preparation or previous manufacture of a unicompartmental interpositional arthroplasty device that comprises a polymeric material such as polyurethane.

In some embodiments, as described below, the present invention therefore provides an implant that is designed to be formed to and congruent with the tibial surface, having a final femoral surface shape that serves largely as a glide path with respect to the femoral condyle. Such a device can be used in patients having joints that have progressed to the stage of “bone on bone,” and thus provides a replacement for the function of articular cartilage, and optionally some of the natural, healthy meniscus, and particularly at the central weight-bearing area, in order to restore alignment, providing an elastomeric, cushioning function. A preferred implant of this type is also congruent with the tibial surface. In turn, the present implant is more permanently anchored in place, in significant part by one or more posterior projections, such as the posterior lip, as well by the optional but preferred use of anterior fixation means (such as, for example, embedded sutures).

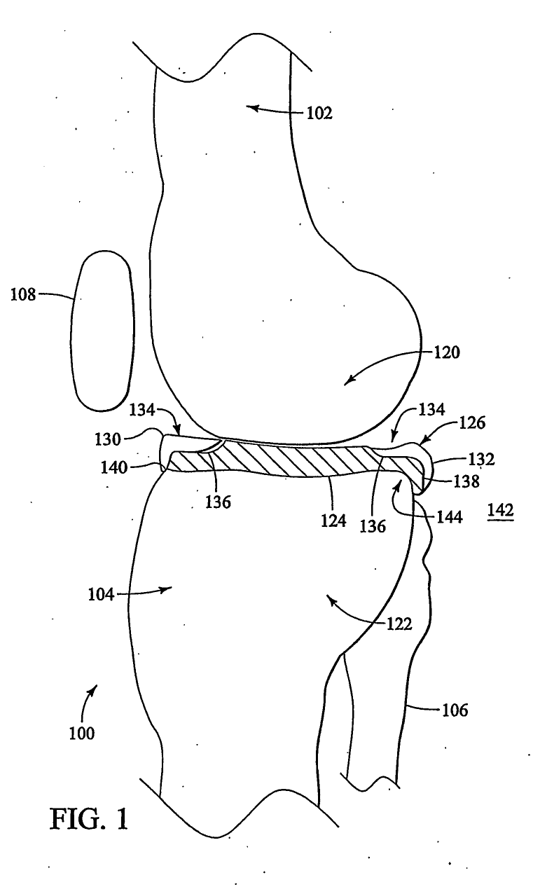

The invention will be further described with respect to the drawings, in which FIG. 1 is a partial medial side view of a right leg 100. Right leg 100 includes a femur 102, a tibia 104, a fibula 106, and a patella 108. Femur 102 includes a medial condyle 120 and tibia 104 includes a tibial plateau 122. An implant 124 in accordance with an exemplary embodiment of the present invention can be seen disposed between medial condyle 120 and tibial plateau 122 in FIG. 1.

A meniscus 126 of right leg 100 is also visible in FIG. 1. In FIG. 1, meniscus 126 is shown being generally disposed about implant 124. Meniscus 126 includes an anterior portion 130 and a posterior portion 132. With reference to FIG. 1, it may be appreciated that meniscus 126 is generally disposed in a cavity 134 defined by a relief 136 of implant 124. In the embodiment of FIG. 1, cavity 134 has a generally annular shape and is disposed proximate an outer perimeter 138 of implant 124.

An implant in accordance with the present invention can be placed in the knee joint by first making an incision 140 at the base of anterior portion 130 of meniscus 126. Implant 124 can then be inserted through incision 140 so as to enter the joint space and reside at least partially within the meniscus. Fixation of implant 124 in situ can be accomplished by effectively capping the tibial plateau with one or more projections extending distally over the rim of the plateau at one end of implant 124 and attaching another end of implant 124 with sutures. In FIG. 1, a posterior lip 142 of implant 124 can be seen extending below a rim 144 of tibia 104.

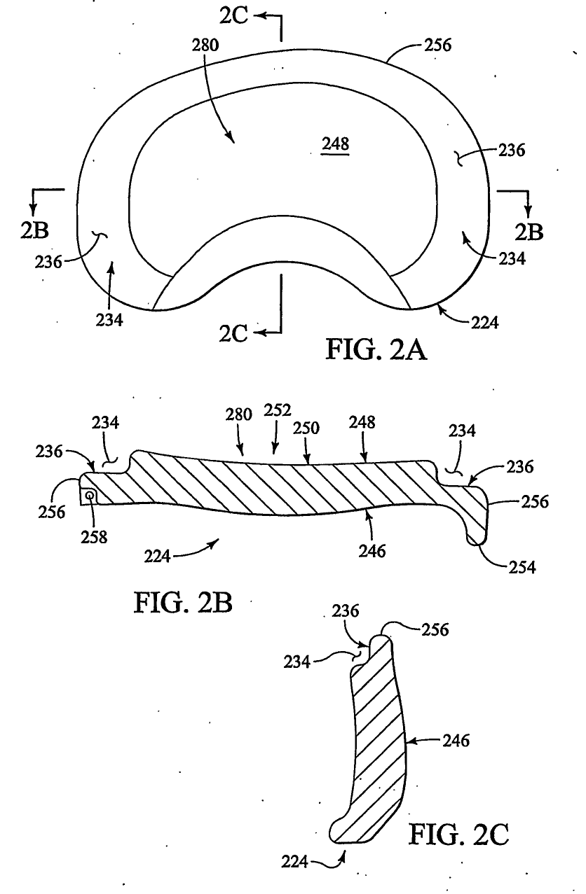

FIG. 2 shows various views of an implant 224 in accordance with an additional exemplary embodiment of the present invention. FIG. 2(a) includes a top view, FIG. 2(b) includes a section view (B-B) taken along section line B-B of the top view, and FIG. 2(c) includes a section view (C-C) taken along section line C-C of view the top view of an embodiment of the invention. Implant 224 comprises a first major surface 246 adapted to be positioned upon the tibial plateau of a tibia, and a second major surface 248 adapted to be positioned against the medial condyle of a femur. In a typical embodiment, second major surface 248 preferably provides a femoral glide path 250 to facilitate its performance in situ, in the form of a generally central depression 252. As shown in FIG. 2, implant 224 also comprises a tibial projection 254, adapted to catch the posterior portion of the tibial plateau by extending over the rim of the tibial plateau distally.

In the embodiment of FIG. 2, implant 224 comprises a relief 236 extending below second major surface 248. With reference to FIG. 2, it may be appreciated that relief 236 defines a cavity 234 having generally annular shape. In some cases, cavity 234 can extend along a circumference 256 of implant 224. In some particularly advantageous embodiments of the present invention, cavity 234 is dimensioned and positioned to receive a meniscus.

Fixation of implant 224 in situ can be accomplished by effectively capping the tibial plateau with tibial projection 254 extending distally over the rim of the plateau at one end of implant 224 and attaching another end of implant 224 with sutures. Implant 224 of FIG. 2 defines a hole 258. In some embodiments of the present invention, hole 258 is dimensioned so as to allow one or more sutures to pass through implant 224. The first major surface 246 of implant 224 provides with a convex bottom configuration in order to better conform to the cavity of an arthritic posterior tibial plateau.

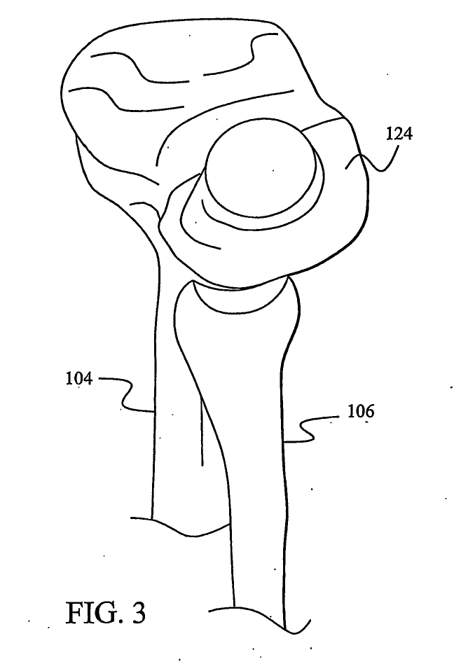

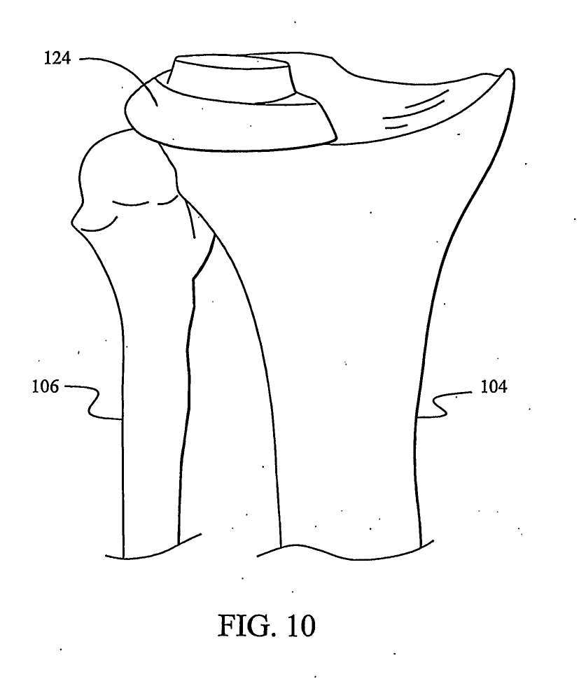

FIG. 3 shows a perspective view a leg, including tibia 104 and fibula 106. An implant 124 in accordance with an exemplary embodiment of the present invention can be seen disposed in the lateral compartment. Implant 124 may be at least partly retained against the tibea 104 by a lateral meniscus (not shown). Implant 124 is also shown in a lateral compartment in FIG. 10.

Implant 124 can comprise features useful for stabilizing it against the tibia 104. As shown in the embodiments in FIGS. 3 and 10, implant 124 can include a skirt useful for stabilizing it against the tibia 104. Such a skirt may be particularly useful for implants designed to be inserted into the lateral compartment. Also as seen in FIGS. 3 and 10, implant 124 can be provided with a pronounced articulating surface. Such a pronounced articulating surface is particularly useful for articulating against the lateral condyle (not shown).

An implant of the type shown provides various benefits, including the correction of varus deformities, based in significant part upon the presence and configuration of the posterior mesial lip, and the cutout (kidney bean shaped) for the intercondylar eminence. The tibial projection is adapted to catch the posterior portion of the tibial plateau. The implant itself has dimensions as provided herein, and can be provided using one of a collection of molds of multiple sizes and/or styles in accordance with the various parameters of the present invention.

Optionally, and preferably, the invention provides an implant that is designed to be formed to and congruent with the tibial surface, having a final femoral surface shape that serves largely as a glide path, such as glide path 280, with respect to the femoral condyle. Such a device can be used in patients having joints that have progressed to the stage of “bone on bone”, and thus provides a replacement for the function of articular cartilage as well as meniscus, and particularly at the central weight-bearing area, in order to restore alignment, providing an elastomeric, cushioning function. A preferred implant of this type is also congruent with the tibial surface, based upon both its initial shape, together with whatever final shaping may occur in situ. In turn, the present implant is more permanently anchored in place, in significant part by one or more posterior projections, such as the posterior lip as well by the optional but preferred use of anterior fixation means (such as embedded sutures).

Such an embodiment includes a unique combination of a femoral glide path and convexity of the tibial surface of the implant, together with a posterior mesial lip. In turn, as provided in the Figures and related description, the implant provides an indentation adapted to accommodate the tibial spine, which together with a slight feathering of the implant on the underside at the tibial spine, the general kidney shape of the implant, and the convexity of the tibial surface, will permit the implant to be congruent with the concave tibia and the posterior mesial lip that extends over the posterior portion of the tibia and into the mesial side of the tibia into the PCL fossa of the tibia. Importantly, such an implant can be provided in various sizes to accommodate different anterior-posterior dimensions of the tibia and different tibial concavities. In other words, the amount of convexity of the tibial surface will be varied with the different sizes depending on the amount of actual concavity that there is in the tibia.

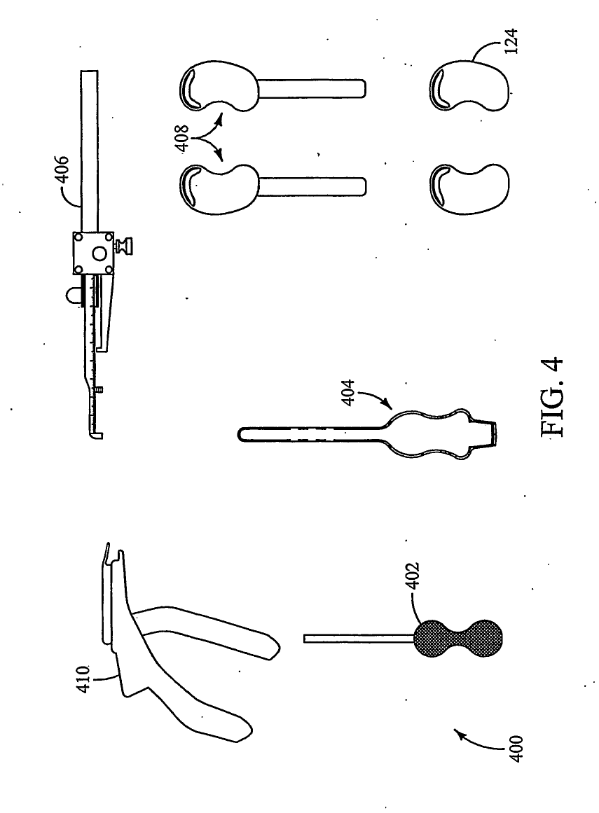

A kit 400 can be supplied for providing implants 124 of various sizes (e.g., implants having thicknesses varying by 1 mm or 2 mm increments and implants having a range of anterior to posterior dimensions), as well as various tools useful for the method of the present invention, as shown in FIG. 4. Implants having different shapes can also be provided (e.g., implants shaped for the lateral compartment and the medial compartment of the left knee and for the right knee).

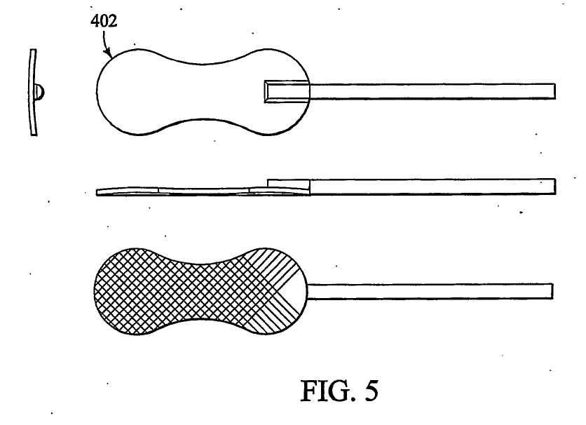

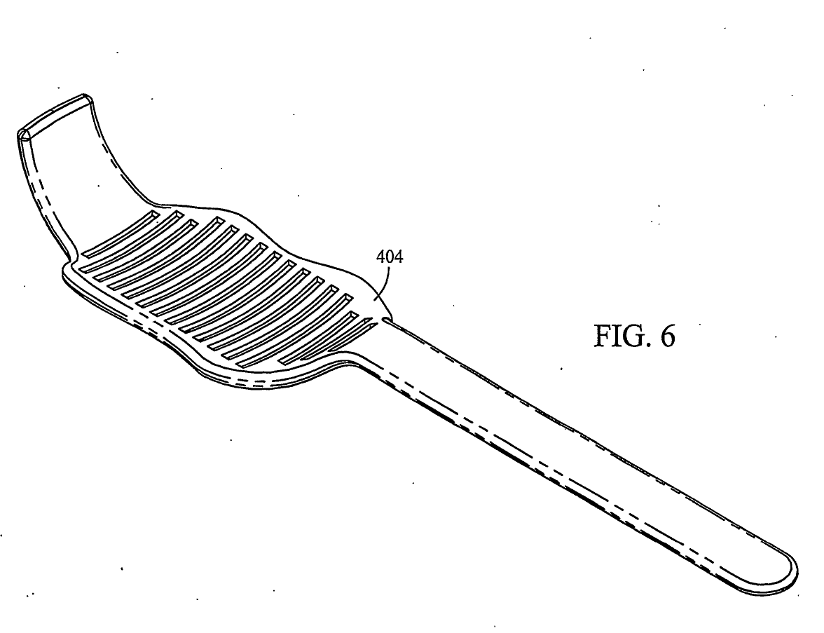

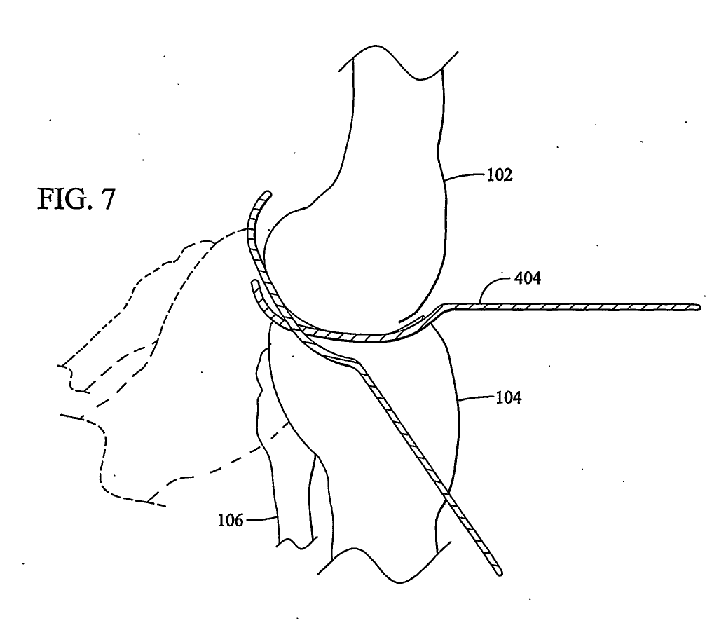

In some embodiments, a range of implant sizes can be provided and sizing can be accomplished by physical measurement using tools and methods as described in International Publication Number WO 2004/006811 A2, the contents of which are herein incorporated by reference. Such an embodiment can include tools for the steps of preparing a joint to receive an implant, including the preparation or resurfacing of a femoral condyle and/or a tibial plateau. For example, a tibial smoother 402 shown in FIG. 5 and a femoral smoother 404 as shown in FIGS. 6 and 7 maybe provided. Femoral smoother 404 can be fenestrated. Such an embodiment is useful for shaving the femur and tibia simultaneously, as well as providing for self cleaning as debris is allowed to pass between the superior and inferior sides. Femoral smoother 404 is preferably universal in its orientation, to permit it to be used in either the right or left leg.

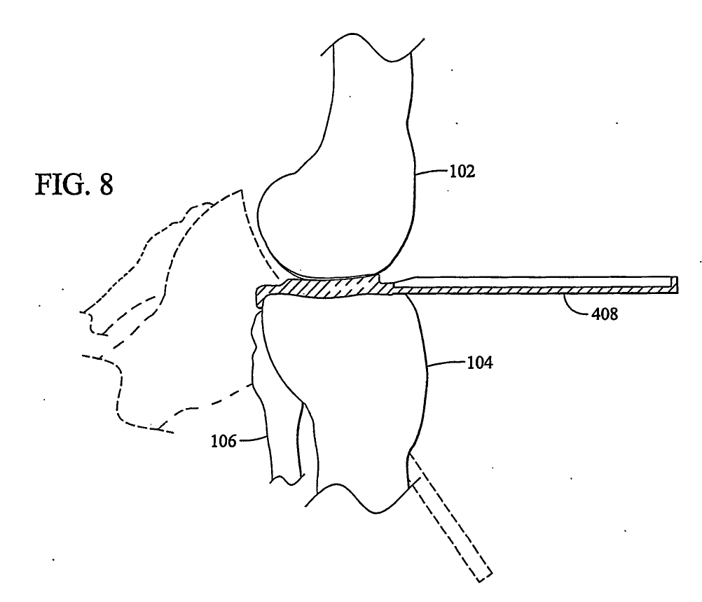

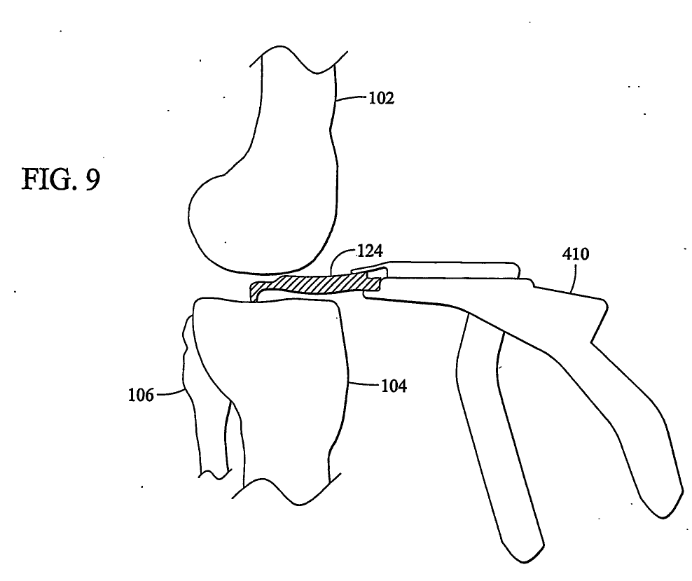

Further, tools can be included for determining an appropriate implant size for a particular joint, such as a sizer 406 as shown in FIG. 4. Tools useful for determining an appropriate implant thickness needed to match physiological values can also be included. For example, one or more implant templates 408 can be included, as shown in FIG. 8. In addition, tools useful for inserting the implant into the joint, such as gripper 410 as shown in FIG. 9, can also be included. Further, the related components and/or devices for performing each step can be included. In such an embodiment, multiple sizes can be made off site and the selection of the appropriate implant size can be chosen at the time of surgery. Alternatively, the pre-made material can be made off site to specifications developed from imaging of the patient's joint surface.

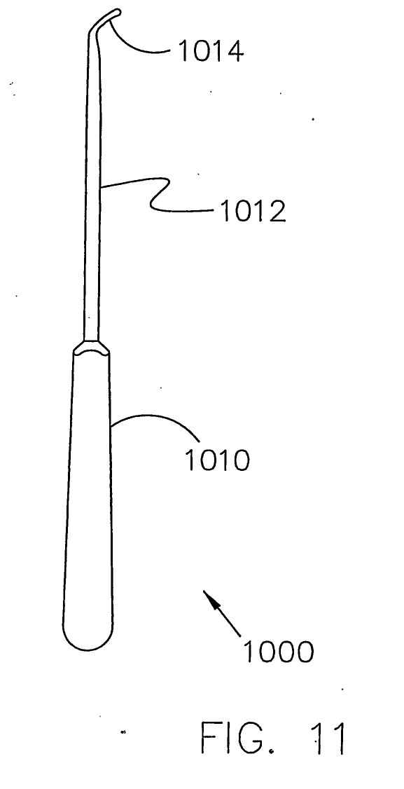

In some embodiments, a rasper 1000 is provided. Rasper 1000 is useful for removing osteophytes, particularly from the posterior region of the tibia plateau. Such removal can be useful for providing congruency between a posterior lip of an implant and the tibia. As shown in FIG. 11, rasper 1000 may comprise a handle 1010, a shaft 1012, and a hook end 1014. The handle 1012 is useful for manipulation of the rasper, and the hook end 1014 can be provided with rasps to remove osteophytes and/or other unwanted debris.

The tools described above can be constructed of any suitable material. For example, the tools can be constructed of stainless steel, ceramic, and/or polymeric materials. Embodiments constructed at least partially of stainless steel can be relatively more suitable for providing a reusable tool, and embodiments constructed at least partially of a polymer can be relatively more suitable for providing a disposable tool. Further, all of the tools above can be shaped to provide an ergonomic fit for the user. Some embodiments provide a universal tool shaped to provide an ergonomic fit for both left and right hands and/or appropriate fit for use in both the right and left knees.

An implant of this invention is preferably used in a method that includes first determining the proper implant thickness needed to match physiological valgus. The surgeon prepares the site arthroscopically, removing excess cartilage while preserving the medial meniscus to the extent possible, using a portal of about 1 cm in order to provide suitable arthroscopic access while maintaining the presence of fluid in the joint. The remaining meniscus can be manipulated to allow for the implant to be placed. The implant can be inserted into the joint. The meniscus can then be placed over and/or on the implant. In some embodiments, the meniscus is placed in apposition to one or more structural features of the implant in a manner that permits the implant to be retained by the meniscus in a manner that further improves the retention of the implant itself upon the tibial surface. The surgeon will then typically feel the implant once in position, to confirm that the implant is properly seated, and will extend the knee to provide varus stress on the lower leg.

Optionally, and preferably, the surgeon can also use femoral forming device 404 (e.g., spoon-shaped device) of the type described in US Provisional Application mailed Dec. 7, 2001 and entitled “Method and Device for Smoothing The Surface of Bone in an Articulating Joint”, the disclosure of which is incorporated herein by reference, in order to prepare a femoral glide path and remove unwanted undulations. The implant can be sutured to the anterior rim, and the knee can be flexed to obtain complete range. Optionally, during or following this procedure, the joint can be filled with a suitable fluid and visualized, after which the knee is extended and braced, with the access portal(s) closed by suitable means (e.g., sutured).

Fixation methods for the implant can include one or more structural features adapted to be fixedly positioned within and/or in apposition to the natural meniscus in a manner that permits the implant to be retained by the meniscus, biologic glues to glue the implant to the underlying surface, trapping of the implant into a cavity on the surface that causes a mechanical lock, using various anchors to the underlying structure and fixing the implant to that surface or using mold retainers and/or screws, staples, sutures or pins. In and alternative embodiment, anchors in the underlying structure can be used for fixing the implant to that surface and/or a tissue ingrowth system can be used to secure anchoring.

In the preferred embodiment, the patient will have a diagnosis of osteoarthritis and have loss of cartilage on the articulating surface. A determination will be made of the amount of correction needed for the reestablishment of a normal angle of articulation. The ligaments will be balanced so that there is no loss of range of motion with the implant in place and the surface will be placed in such a position that the eventual resulting surface geometry reestablishes the same plane and orientation of the original articular surface.

Access to the site is obtained in a minimally invasive way. In some embodiments, this is accomplished through arthroscopic means with arthroscopic portals. In an alternative embodiment, the access is accomplished by a mini arthrotomy with a small incision that allows access to the joint without sacrificing nerves, vessels, muscles or ligaments surrounding the joint. In the preferred embodiment fibrillated articulating cartilage that is degenerated is removed down to the subchondral surface. The surface is dried and prepared for appropriate anchoring. This can include anchor points that give a mechanical lock or that alternatively simply supply horizontal and lateral stability. The surface can be prepared by drying and roughening in case a tissue adhesive is used. Pre-made anchors can be installed. These can be made of various metallic materials or of polymers and can consist of pegs that can extend up through the implant to anchor it to the underlying surface. This surrounding subchondral bone can be roughened to enhance tissue ingrowth or implant adhesion.

Various forms of stabilization can be used, including anchor points or titanium screws. Alternatively, the pre-made material can be made off site to the specifications developed from imaging of the patient's joint surface. In a third embodiment, multiple sizes can be made off site and the selection of the appropriate implant size can be chosen at the time of surgery. Further, the implant can comprise several segments and a single segment can be installed through a portal or a series of segments can be installed through a portal and locked together once inside the joint. They can be placed sequentially and then anchored to the bone by anchor points cut in the bone or by screws or tissue ingrowth. Finally, a robot, a jag or other cutting fixture can be used to prepare the bony surface for the pre-made implant to a fixed geometry of the anchor point.

The biomaterial can be prepared from any suitable material. Generally, a material is suitable if it has appropriate biostability, biodurability and biocompatibility characteristics. Typically, the materials include polymeric materials, having an optimal combination of such properties as biostability, biodurability, biocompatibility, physical strength and durability, and compatibility with other components (and/or biomaterials) used in the assembly of a final composite.

Examples of polymeric materials that may be suitable in some applications, either alone or in combination, include polyurethane, available from Polymer Technology Group Incorporated under the names Bionate,™ Biospan,™ and Elasthane™, available from Dow Chemical Company under the name Pellethane,™ and available from Bayer Corp. under the names Bayflex,™ Texin,™ and Desmopan;™ ABS, available from GE Plastics under the name Cycolac™, and available from Dow Chemical Company under the name Magnum;™ SAN, available from Bayer Plastics under the name Lustran;™ Acetal, available from Dupont under the name Delrin,™ and available from Ticona GmbH and/or Ticona LLC (Ticona) under the name Celcon;™ polycarbonate, available from GE Plastics under the name Lexan,™ and available from Bayer Corp. under the name Makrolon;™ polyethylene, available from Huntsman LLC, and available from Ticona under the names GUR 1020™ and GUR 1050;™ polypropylenes, available from Solvay Engineered Polymers, Inc. under the name Dexflex;™ aromatic polyesters, available from Ticona; polyetherimide (PEI), and available from GE Plastics under the name Ultem;™ polyamide-imide (PAI), available from DSM E Products under the name Torlon;™ polyphenylene sulfide, available from Chevron Phillips Chemical Company LP under the name Ryton;™ polyester, available from Dupont under the name Dacron;™ polyester thermoset, available from Ashland Specialty Chemical Company under the name Aropol;™ polyureas; hydrogels, available from Hydromer Inc.; liquid crystal polymer, available from Ticona under the name Vectra;™ polysiloxanes, available from Nusil Technologies, Inc.; polyacrylates, available from Rohm & Haas under the name Plexiglas;™ epoxies, available from Ciba Specialty Chemicals; polyimides, available from Dupont under the names Kapton,™ and Vespel;™ polysulfones, available from BP Amoco Chemicals under the name Udel,™ and available from BASF Corporation under the name Ultrason;™ PEAK/PEEK, available from Victrex under the name Victrex PEAK;™ as well as biopolymers, such as collagen or collagen-based materials, chitosan and similar polysaccharides, and combinations thereof. Of course, any of the materials suitable for use in a composite or single biomaterial implant may be structurally enhanced with fillers, fibers, meshes or other structurally enhancing means.

The present invention provides a biomaterial having an improved combination of properties for the preparation, storage, implantation and long term use of medical implants. The improved properties correspond well for the preparation and use of an implant having both weight bearing and/or articulating functions, and preferably in the form of an implant for interpositional arthroplasty.

In turn, a preferred biomaterial of this invention provides an optimal combination of properties relating to wear resistance, congruence, and cushioning while meeting or exceeding requirements for biocompatibility, all in a manner that serves to reduce the coefficient of friction at the major motion interface.

Wear resistance can be assessed by determining parameters such as DIN abrasion and flexural stress strain fatigue resistance. A preferred implant will have sufficient wear resistance to avoid the generation of clinically significant particulate debris over the course of the implant's use.

Congruence can be assessed by determining parameters such as tensile modulus compressive modulus, and hardness, to determine the manner and extent to which the implant will conform itself to possible other components of the implant itself and/or to bone or surrounding tissue.

Cushioning can be assessed by determining such parameters as hardness, compressive modulus, and tensile modulus, to determine the elastomeric nature of the material, and in turn, its suitability for use in a weight bearing joint. More elastomeric materials will generally provide greater comfort in weight bearing applications, particularly if the other physical properties can be maintained.

Applicant has discovered that improved wear resistance, congruence, and/or cushioning toughness can be achieved without undue effect on other desired properties, such as abrasion, hardness, specific gravity, tear resistance, tensile strength, ultimate elongation, and biocompatibility. Moreover, Applicant has discovered that such properties can themselves be provided in varying forms, as between first and second biomaterials of a composite of the present invention.

A polymeric biomaterial of this invention can be prepared using any suitable means, including by curing the polymer ex vivo. The composition can be used in any suitable combination with other materials, including other compositions of the same or similar nature, as well as other materials such as natural or synthetic polymers, metals, ceramics, and the like.

The invention further provides a method of preparing the composition, a method of using the composition, implants that comprise the composition, as well as methods of preparing and using such implants.

The biomaterial used in this invention preferably includes polyurethane components that are reacted ex vivo to form a polyurethane (“PU”). The formed PU, in turn, includes both hard and soft segments. The hard segments are typically comprised of stiffer oligourethane units formed from diisocyanate and chain extender, while the soft segments are typically comprised of one or more flexible polyol units.

These two types of segments will generally phase separate to form hard and soft segment domains, since they tend to be incompatible with one another. Those skilled in the relevant art, given the present teaching, will appreciate the manner in which the relative amounts of the hard and soft segments in the formed polyurethane, as well as the degree of phase segregation, can have a significant impact on the final physical and mechanical properties of the polymer. Those skilled in the art will, in turn, appreciate the manner in which such polymer compositions can be manipulated to produce cured and curing polymers with desired combination of properties within the scope of this invention.

The hard segments of the polymer can be formed by a reaction between the diisocyanate or multifunctional isocyanate and chain extender. Some examples of suitable isocyanates for preparation of the hard segment of this invention include aromatic diisocyanates and their polymeric form or mixtures of isomers or combinations thereof, such as toluene diisocyanates, naphthalene diisocyanates, phenylene diisocyanates, xylylene diisocyanates, and diphenylmethane diisocyanates, and other aromatic polyisocyanates known in the art. Other examples of suitable polyisocyanates for preparation of the hard segment of this invention include aliphatic and cycloaliphatic isocyanates and their polymers or mixtures or combinations thereof, such as cyclohexane diisocyanates, cyclohexyl-bis methylene diisocyanates, isophorone diisocyanates and hexamethylene diisocyanates and other aliphatic polyisocyanates. Combinations of aromatic and aliphatic or arylakyl diisocyanates can also be used.

The isocyanate component can be provided in any suitable form, examples of which include 2,4′-diphenylmethane diisocyanate, 4,4′-diphenylmethane diisocyanate, and mixtures or combinations of these isomers, optionally together with small quantities of 2,2′-diphenylmethane diisocyanate (typical of commercially available diphenylmethane diisocyanates). Other examples include aromatic polyisocyanates and their mixtures or combinations, such as are derived from phosgenation of the condensation product of aniline and formaldehyde. It is suitable to use an isocyanate that has low volatility, such as diphenylmethane diisocyanate, rather than more volatile materials such as toluene diisocyanate. An example of a particularly suitable isocyanate component is the 4,4′-diphenylmethane diisocyanate (“MDI”). Alternatively, it can be provided in liquid form as a combination of 2,2′-, 2,4′- and 4,4′-isomers of MDI. In a preferred embodiment, the isocyanate is MDI and even more preferably 4,4′-diphenylmethane diisocyanate.

In one embodiment of the invention, the isocyanate is 4,4′-diphenylmethane diisocyanate (as available from Bayer under the tradename Mondur M), from preferably about 20 to 60 weight percent, more preferably from about 30 to 50 weight percent. The actual amount of isocyanate used should be considered in combination with other ingredients and processing parameters, particularly including the amount of chain extender (such as butanediol (BDO)) used, since the combination typically determines the hard segment component, and in turn hardness, of the corresponding cured polymer. Hardness correlates in a generally proportional fashion with the combined weights of MDI and BDO, such that compositions having between 30 and 60 total weight percent (MDI+BDO) are generally useful, with those compositions having between about 50 to about 60 total weight percent being somewhat harder, and particularly useful for either the first (femoral contacting) biomaterial and surface of a composite implant or for implants having a single biomaterial providing both first and second surfaces. By contrast, compositions having between about 40 to about 50 total weight percent are somewhat more congruent and cushioning, though less wear resistant, and therefore are preferred for use as the second biomaterial, e.g., tibial contacting surface, of a composite implant as described herein.

Some examples of chain extenders for preparation. of the hard segment of this invention include, but are not limited, to short chain diols or triols and their mixtures or combinations thereof, such as 1,4-butane diol, 2-methyl-1,3-propane diol, 1,3-propane-diol ethylene glycol, diethylene glycol, glycerol, tri-methylpropane, cyclohexane dimethanol, triethanol amine, and methyldiethanol amine. Other examples of chain extenders for preparation of the hard segment of this invention include, but are not limited to, short chain diamines and their mixtures or combinations thereof, such as dianiline, toluene diamine, cyclohexyl diamine, and other short chain diamines known in the art.

The soft segment consists of urethane terminated polyol moieties, which are formed by a reaction between the polyisocyanate or diisocyanate or polymeric diisocyanate and polyol. Examples of suitable diisocyanates are denoted above. Some examples of polyols for preparation of the soft segment of this invention include but are not limited to polyalkylene oxide ethers derived form the condensation of alkylene oxides (e.g. ethylene oxide, propylene oxide, and blends thereof), as well as tetrahyrofuran based polytetramethylene ether glycols, polycaprolactone diols, polycarbonate diols and polyester diols and combinations thereof. In a preferred embodiment, the polyols are polytetrahydrofuran polyols (“PTHF”), also known as polytetramethylene oxide (“PTMO”) or polytetramethylene ether glycols (“PTMEG”). Even more preferably, the use of two or more of PTMO diols with different molecular weights selected from the commercially available group consisting of 250, 650, 1000, 1400, 1800, 2000 and 2900.

Two or more PTMO diols of different molecular weight can be used as a blend or separately, and in an independent fashion as between the different parts of a two part system. The solidification temperature(s) of PTMO diols is generally proportional to their molecular weights. The compatibility of the PTMO diols with such chain extenders as 1,4-butanediol is generally in the reverse proportion to the molecular weight of the diol(s). Therefore the incorporation of the low molecular weight PTMO diols in a “curative” (part B) component of a two part system, and higher molecular weight PTMO diols in the prepolymer (part A) component, can provide a two-part system that can be used at relatively low temperature. In turn, good compatibility of the low molecular weight PTMO diols with such chain extenders as 1,4-butanediol permits the preparation of two part systems with higher (prepolymer to curative) volume ratio. Amine terminated polyethers and/or polycarbonate-based diols can also be used for building of the soft segment.

In one embodiment of the invention, the polyol is polytetramethyleneetherglycol 1000 (as available from E.I. du Pont de Nemours and Co. under the tradename Terathane 1000), preferably from about 0 to 40 weight percent, more preferably from about 10 to 30 weight percent, and perhaps even more preferably from about 22 to 24 weight percent, based on the total weight of the polymer. The polyol disclosed above may be used in combination with polytetramethyleneetherglycol 2000 (as available from E.I. du Pont de Nemours and Co. under the tradename Terathane 2000), preferably from about 0 to 40 weight percent, more preferably from about 10 to 30 weight percent, and perhaps even more preferably from about 17 to 18 weight percent, based on the total weight of the polymer.

In one embodiment, the biomaterial may include a chain extender. For example, the chain extender may be 1,4-butanediol (as available from Sigma Aldrich Corp.), preferably from about 1 to 20 weight percent, more preferably from 5 to 15 weight percent, to perhaps even more preferably from 12 to 13 weight percent, based on the total weight of the polymer.

The polyurethane can be chemically crosslinked, e.g., by the addition of multifimctional or branched OH-terminated crosslinking agents or chain extenders, or multifunctional isocyanates. Some examples of suitable crosslinking agents include, but are not limited to, trimethylol propane (“TMP”), glycerol, hydroxyl terminated polybutadienes, hydroxyl terminated polybutadienes (HOPB), trimer alcohols, Castor oil polyethyleneoxide (PEO), polypropyleneoxide (PPO) and PEO-PPO triols. In a preferred embodiment, HOPB is used as the crosslinking agent.

This chemical crosslinking augments the physical or “virtual” crosslinking of the polymer by hard segment domains that are in the glassy state at the temperature of the application. The optimal level of chemical cross-linking improves the compression set of the material, reduces the amount of the extractable components, and improves the biodurability of the PU. This can be particularly useful in relatively soft polyurethanes, such as those suitable for the repair of damaged cartilage. Reinforcement by virtual cross-links alone may not generate sufficient strength for in vivo performance in certain applications. Additional cross-linking from the soft segment, potentially generated by the use of higher functional polyols can be used to provide stiffer and less elastomeric materials. In this manner a balancing of hard and soft segments, and their relative contributions to overall properties can be achieved.

In one embodiment, the chemical cross-linking agent is 2-ethyl-2-(hydroxymethyl)-1,3-propanediol (also known as trimethylolpropane, as available from Sigma Aldrich Corp.), preferably from about 0 to 5 weight percent, more preferably from about 0.1 to 1 weight percent, and perhaps even more preferably from about 0.15 to 0.3 weight percent, based on the total weight of the polymer.

Additionally, and optionally, a polymer system of the present invention may contain at least one or more biocompatible catalysts that can assist in controlling the curing process, including the following periods: (1) the cure induction period, and (2) the full curing period of the biomaterial. Together these two periods, including their absolute and relative lengths, and the rate of acceleration or cure within each period, determine the cure kinetics or profile for the composition. In some embodiments, however, a catalyst is not included. For instance embodiments in which the biomaterial is heated in the course of curing, such as in a heated mold in the manner described herein, can performed without the use of a catalyst.

Some examples of suitable catalysts for preparation of the formed PU of this invention include, but are not limited to, tin and tertiary amine compounds or combinations thereof such as dibutyl tin dilaurate (DBTDL), and tin or mixed tin catalysts including those available under the tradenames “Cotin 222”, “Fomrez UL-22” (Crompton Corp.), “dabco” (a triethylene diamine from Sigma-Aldrich), stannous octanoate, trimethyl amine, and triethyl amine.

In one embodiment of the invention, the catalyst is bis-(dodecylthio)-dimethylstannane (available from Crompton Corp. as Fomrez catalyst UL-22), preferably from about 0 to 2 weight percent, more preferably from about 0 to 1 weight percent, and perhaps most preferably from 0.0009 to 0.002 weight percent, based on the total weight of the polymer.

Further, a polymer stabilizer additive useful for protecting the polymer from oxidation may be included. In one embodiment of the invention, the additive is pentaerythritol tetrakis (3-(3,5-di-tert-buyl-4-hydroxyphenyl)proprionate (available from Ciba Specialty Chemicals, Inc. as Irganox 1010), preferably from about 0 to 5 weight percent, more preferably about 0.1 to 1 weight percent, and perhaps even more preferably about 0.35 to 0.5 weight percent, based on the total weight of the polymer.

Optionally, other ingredients or additives can be included, for instance, a reactive polymer additive can be included from the group consisting of hydroxyl- or amine-terminated compounds selected from the group consisting of poybutadiene, polyisoprene, polyisobutylene, silicones, polyethylene-propylenediene, copolymers of butadiene with acryolnitrile, copolymers of butadiene with styrene, copolymers of isoprene with acrylonitrile, copolymers of isoprene with styrene, and mixtures of the above. Other additives may also be optionally provided. For example, catalysts such as Dabco, antioxidants such as vitamin E, hydrophobic additives such as hydroxyl-terminated polybutadiene, and dye green GLS, singularly or in combination, may be included in the polymer formulation.

Suitable compositions for use in the present invention are those polymeric materials that provide an optimal combination of properties relating to their manufacture, application, and in vivo use. In the uncured state, such properties include component miscibility or compatibility, processability, and the ability to be adequately sterilized or aseptically processed and stored. While the composition is curing, suitable materials exhibit an optimal combination of cure kinetics and exotherm. In the cured state, suitable compositions exhibit an optimal combination of such properties as abrasion, hardness, specific gravity, tear resistance, tensile strength, ultimate elongation, and biocompatibility.

The composition of the present invention provides a polyurethane that can be prepared ex vivo. Particularly when formed ex vivo, products incorporating the composition of this invention may be made in advance of their use, on a commercial scale, and under stringent conditions.

Polymeric biomaterials of this invention, including preferred polyurethanes can be prepared using automated manufacturing processes within the skill of those in the art. A preferred manufacturing method, for instance, includes the use of multichannel dispensing equipment to inject the polymer. Such equipment is well suited to high precision applications, having a variable or fixed number of channels, some have all channels dispensing the same volume while in others the volume can be set by channel, some have all channels dispensing the same fluid, while others allow for different fluids in different channels. The dispensing can be automated repetitive or manual. Suitable devices for metering, mixing and dispensing materials such as urethanes are commercially available from a variety of sources, including for instance from Adhesive Systems Technology Corp., 9000 Science Center Drive, New Hope, Minn. 55428.

Furthermore, polymeric biomaterials of this invention may be cured in a heated mold. The mold may receive the contents of the polymeric biomaterial before it is cured. In one embodiment, a permanent enclosed mold is used to form at least a part of the implant. Such a mold may be similar to a standard injection mold and have the ability to withstand large clamping forces. Further, such a mold may include runners and/or vents to allow material to enter and air to exit. Such a mold may be constructed from metals, polymers, ceramics, and/or other suitable materials. The mold may be capable of applying and controlling heat to the biomaterial to accelerate curing time. In some embodiments, the mold may be coated with a release coating agent to facilitate ease of removal of the cured biomaterial from the mold. Examples of suitable release agents include Teflon,™ silicone, florinated ethylene propylene (FEP), dichronite, gold, and nickel-Teflon combinations, various types of which are commercially available from a variety of sources, e.g., McLube Division of McGee Industries. In addition, the mold may be provided in two separable parts to further facilitate removal of the cured biomaterial.

Further, time and temperature parameters can be modified in processing to change the characteristics of the implant. A time temperature profile may be selected to achieve certain implant properties. In embodiments formed with a heated mold as described above, those skilled in the art will appreciate the manner in which both the temperature of the mold as well as the time biomaterial is maintained can be adjusted to change the characteristics of the molded implant.

In the embodiment in which an ex vivo curing polymer is used, the present invention preferably provides a biomaterial in the form of a curable polyurethane composition comprising a plurality of parts capable of being at least partially mixed at a time before use, the parts including: (1) a polymer component comprising the reaction product of one or more polyols, and one or more diisocyanates, and (2) a curative component comprising one or more chain extenders, one or more catalysts, and optionally, one or more polyols and/or other optional ingredients.

In some embodiments, long term congruence of the biomaterial is facilitated by its hydration in vivo, permitting the biomaterial to become more pliable, and in turn, facilitate congruence with the tibial plateau. In turn, an increase in hydration and/or changes in temperature can improve the fit and mechanical lock between the implant and the tibial plateau. The biomaterial may be hydrated ex vivo and/or in vivo, both before and after the composition is cured. Preferably, the biomaterial may be further hydrated within the joint site after the composition in order to enhance both conformance and performance of the implant.

Implantable compositions of this invention demonstrate an optimal combination of properties, particularly in terms of their physical/mechanical properties, and biocompatibility. Such performance can be evaluated using procedures commonly accepted for the evaluation of natural tissue, as well as the evaluation of materials and polymers in general. In particular, a preferred composition, in its cured form, exhibits physical and mechanical properties that approximate or exceed those of the natural tissue it is intended to provide or replace. Fully cured polymeric (e.g., polyurethane) biomaterials within the scope of this invention provide an optimal combination of such properties as abrasion, compressive hardness, compressive modulus hardness, specific gravity, tear resistance, tensile strength, ultimate elongation, tensile modulus, and biocompatibility.

Physical/Mechanical Properties and Test Methods

Various properties of the composition of this invention can be evaluated for use in quality control, for predicting service performance, to generate design data, to determine compliance with established standards, and on occasion, to investigate failures. See, for instance, Handbook of Polymer Testing: Physical Methods, edited by Roger Brown, Marcel Dekker, Inc., New York, N.Y. (1999), the disclosure of which is incorporated herein by reference. Suitable properties include those dealing with a) mass, density and dimensions, b) processability, c) strength and stiffness (including compressive hardness, compressive modulus, tensile stress-strain, flexural stress-strain, flexibility, and tear tests), c) fatigue and wear (including abrasion resistance and hardness), d) time dependent properties (such as creep, stress relaxation, compression set, tension set), e) effect of temperature (such as thermal expansion, shrinkage, and thermal oxidative aging), f) environmental resistance, and g) and biocompatibility parameters.

Of particular note are those properties that lend themselves to the preparation, delivery and long term use of improved implants having an articulating surface, and preferably for long term weight bearing use.

The preferred property ranges given below are only relevant to certain embodiments of the invention. It will be appreciated by those reasonably skilled in the art that materials having one or more properties outside the scope of the preferred ranges given below are suitable for use with the present invention.

Abrasion values for a polymer can be determined with a rotating cylindrical drum device, known as a DIN abrader. A loaded cylindrical test piece is traversed along an abrasive cloth attached to a rotating drum, and the mass loss is measured after a specified length of travel. Advantages of this device include the use of a test piece small enough to be cut from a product or a comparatively thin sheet and a much reduced risk of abrasive contamination caused by debris or smearing. The result can be expressed with the abrasion resistance index, which is the ratio of the volume loss of a black standard rubber sample to the volume loss of the test sample.

The polymer preferably provides a DIN abrasion value of less than about 70 mm³, more preferably less than about 60 mm³ and most preferably less than about 50 mm³, as determined by ASTM Test Method D5963-96 (“Standard Test Method for Rubber Property Abrasion Resistance Rotary Drum Abrader”). DIN abrasion values of greater than about 70 mm³ tend to exhibit wear rates that are too great for longer term use as articulating surface.

Biomaterial can be formed into standardized (e.g., puck-like) implant shapes and subjected to conditions intended to replicate, while also meet and exceed physiological conditions. Preferred biomaterials of this invention are able to withstand one million cycles (approximately equivalent to 1 year implantation), and more preferably greater than 5 million cycles (approximately equivalent to 5 years) before generating unsuitable debris.

Flexural stress/strain fatigue can be measured in a variety of ways. Using the standardized shape as described above, samples can be compressively loaded in cycles of increasing loads, and the stress strain fatigue can be plotted verses the number of cycles.

As another example, flexural stress/strain fatigue can be determined by a three point bending test, in which a standardized implant sample shape is supported at its anterior and posterior ends. A cyclical load is applied to the sample in an area substantially between the two supports to provide a deflection of approximately 4 mm, and the total number of cycles until failure is recorded.

Biomaterials formed into implant shapes in accordance with the present invention, under conditions intended to meet and exceed physiological conditions, are preferably able to withstand one million cycles (approximately equivalent to 1 year implantation), and more preferably greater than five million cycles (approximately equivalent to 5 years implantation) in a test similar to the one described above.

Fracture toughness can generally be determined by a number of methods. For example, fracture toughness can be measured by tests similar to ASTM Test Method D5045-99.

Preferably, the polymer provides a peak load fracture toughness of at least about 50 lbs, more preferably more than about 80 lbs, and most preferably more than about 110 lbs. Further, the polymer preferably provides an energy to break fracture toughness of greater than about 15 lb-in, more preferably greater than about 25 lb-in, and most preferably greater than about 30 lb-in. These values may be obtained with tests similar to ASTM Test Method D5045-99.

The term hardness has been applied to scratch resistance and to rebound resilience, but for polymers it is taken to refer to a measure of resistance to indentation. The mode of deformation under an indentor is a mixture of tension, shear, and compression. The indenting force is usually applied in one of the following ways: Application of a constant force, the resultant indentation being measured, measurement of the force required to produce a constant indentation, or use of a spring resulting in variation of the indenting force with depth of indentation.

A biomaterial of this invention preferably provides a hardness value when hydrated of less than about 75 Shore D, more preferably less than about 70 Shore D, and most preferably less than about 60 Shore D, as determined by ASTM Test Method D2240. In some embodiments, hydration of the biomaterial may lower the shore hardness value.

In one method of determining specific gravity, a test piece is provided weighing a minimum of 2.5 grams, which can be of any shape as long as the surfaces are smooth and there are no crevices to trap air. The test piece is weighed in air and then in water using a balance accurate to 1 mg. The test piece can be suspended by means of a very fine filament, the weight of which can be included in the zero adjustment of the balance and its volume in water ignored. The specific gravity is calculated from the difference in measurements.

The polymer preferably provides a specific gravity of about 1 to 2 g/cm³, more preferably about 1 to 1.5 g/cm³, and most preferably about 1.15 to 1.17 g/cm³, as determined by ASTM Test Method D792.

A tear test may be used to measure tear strength. In a tear test, the force is not applied evenly but is concentrated on a deliberate flaw or sharp discontinuity in the sample and the force to produce continuously new surface is measured. This force to start or maintain tearing will depend in a complex manner on the geometry of the test piece and the nature of the discontinuity.

Preferably, a biomaterial of this invention provides a tear strength value in the Die C configuration of greater than about 400 pounds per linear inch (PLI), more preferably greater than about 600 PLI, and most preferably greater than about 800 PLI, and a value in the Die T configuration of preferably greater than about 100 PLI, more preferably greater than about 150 PLI, and most preferably greater than about 250 PLI, as determined by ASTM Test Method D624.

To measure tensile modulus, tensile strength, and ultimate elongation, a test piece of the material is stretched until it breaks, and the force and elongation at various stages is measured. A tensile machine is used to perform this test. Generally, the basic elements of a tensile machine are grips to hold the test piece, a means of applying a strain (or stress), a force-measuring instrument, and an extensometer.

The polymer preferably provides a tensile modulus at 100% elongation value of about 1,000 to 10,000 psi, more preferably about 2,000 to 5,000 psi, and most preferably about 2,500 to 4,500 psi, as determined by ASTM Test method D412.

The polymer preferably provides a tensile modulus at 200% elongation value of about 1,000 to 10,000 psi, more preferably about 2,000 to 6,000 psi, and most preferably about 2,500 to 5,000 psi, as determined by ASTM Test method D412.

The polymer preferably provides a tensile strength value of greater than about 6,000 psi, more preferably greater than about 6,500 psi, and most preferably greater than about 7,000 psi., as determined by ASTM Test Method D412.

Preferably, the polymer provides an ultimate elongation of greater than about 200%, more preferably greater than about 250%, and most preferably greater than about 300%, as determined by ASTM Test Method D412.

To measure compressive modulus and compressive strength, a sample is again formed in a standardized (e.g., puck) shape and varying compressive loads are applied to the sample in order to develop a corresponding curve. The compressive modulus can be determined from this curve. Compressive strength may be determined by applying increasing loads to a sample until the sample fails.

Preferably, the sample implant provides an compressive modulus of greater than about 4,000 psi, more preferably greater than about 4,500 psi, and most preferably greater than about 5,000 psi, as determined in the manner described above.

Preferably, the sample implant also provides a compressive strength of greater than about 6,000 psi, more preferably greater than about 7,000 psi, and most preferably greater than about 8,000 psi, as determined by a test similar to the one described above.

Water absorption may be determined in a variety of ways. A suitable method for measuring water absorption is to submerge a sample of the test material, with an implant-type geometry, in a saline solution. Once the sample and saline solution reach equilibrium at 37 degrees Celsius, which may take a month or longer, the sample is removed and weighed to determine its water absorption.

Preferably, the polymer provides a water absorption value less than about 5% at 37 C, more preferably less than about 3% at 37 C, and most preferably less than about 2% at 37 C, as determined by a test similar to the one described above.

The medical-grade polyurethane resins were evaluated for biocompatibility in accordance with ISO 10993: Biological Evaluation of Medical Devices and FDA G95-1: Required Biocompatibility Training and Toxicology Profiles for Evaluation of Medical Devices. The biological effects of the resin, such as cytotoxicity, sensitization, genotoxicity, implantation, chronic toxicity, and carcinogenicity, were studied. The tests were conducted in accordance with the FDA Good Laboratory Practice (GLP) Regulation.

The following tests were conducted to determine if the polymer is biocompatible: 1) ISO MEM elution using L-929 mouse fibroblast cells; 2) ISO agarose overlay using L-929 mouse fibroblast cells; 3) ISO acute systemic injection test; 4) ISO intracutaneous reactivity test; 5) ISO guinea pig maximization sensitization test; 6) Material mediated rabbit pyrogen test; 7) In vitro genotoxicology test; and 8) ISO muscle implantation study in the rabbit with histology-1 week. The results of the eight selected screening biocompatibility tests above show that the polymer passes all the tests and is considered biocompatible.

In an alternative embodiment, the implant can be provided by any of a series of metals, including titanium, stainless steel, cobalt chrome millithium alloys and tantalum. Other surface materials can include various ceramics and biologic polymers.

Numerous characteristics and advantages of the invention covered by this document have been set forth in the foregoing description. It will be understood, however, that this disclosure is, in many respects, only illustrative. Changes may be made in details, particularly in matters of shape, size and ordering of steps without exceeding the scope of the invention. The invention's scope is, of course, defined in the language in which the appended claims are expressed.

What is claimed is:

1. An interpositional arthrosplasty implant adapted to be retained in position in apposition to a joint surface, at least in part, by surrounding healthy tissue.

2. An implant according to claim 1 wherein the implant comprises a knee implant and includes one or more structural features adapted to be fixedly positioned within and/or in apposition to the natural meniscus, in a manner that permits the implant to be retained by the meniscus in a manner that further improves the retention of the implant itself upon the tibial surface.

3. An implant according to claim 1 wherein the implant comprises a polymeric component comprising a first major surface adapted to be positioned upon and congruent with the tibial surface of the knee, and a second major surface adapted to be positioned against the femoral condyle of the knee.

4. An implant according to claim 3 wherein the implant includes a relief defining a cavity extending below the second major surface.

5. An implant according to claim 4 wherein the cavity extends along at least a portion of a circumference of the implant, and is adapted to be retained within the meniscal tissue of a knee.

6. An implant according to claim 2 wherein the implant is selected from the group consisting of medial implants and lateral implants.

7. An implant in accordance with claim 3 wherein the implant comprises a biomaterial.

8. An implant in accordance with claim 7 wherein the biomaterial comprises a polyurethane.

9. An implant according to claim 8 wherein the polyurethane is biocompatible with respect to cytotoxicity, sensitization, genotoxicity, chronic toxicity, and carcinogenicity.

10. An implant according to claim 9 wherein polyurethane has a Shore hardness of at least about 60 D or less.

11. A kit for a positional arthroplasty system, the kit comprising:
a) an implant that provides a major surface adapted to be positioned against a femoral condyle and a second major surface adapted to be positioned against a tibial plateau, and
b) one or more devices adapted to perform one or more steps selected from the group consisting of preparing the joint to receive an implant, determining an appropriate implant size for a particular joint, inserting the implant into the joint, and/or securing the implant to a desired extent.

12. A kit according to claim 11 wherein the kit includes a includes an femoral smoother.

13. A kit according to claim 12 wherein the femoral smoother is universal in its orientation.

14. A kit according to claim 12 wherein the femoral smoother is fenestrated.

15. A kit according to claim 11 wherein the kit includes one or more trial implants.

16. A method of repairing a knee joint, comprising the steps of providing and implanting an implant according to claim 1.

17. A knee joint that includes an implant according to claim 1.

18. A kit comprising a tool useful for preparing a joint to receive an implant, an apparatus useful for determining an appropriate implant size for the joint, an apparatus useful for determining an appropriate implant thickness, and a tool useful for inserting the implant into the joint and/or securing the implant to a desired extent.

21. An implant according to claim 1 wherein the implant comprises a composite or monolith structure fabricated from a biocompatible, biodurable material that is adapted to be inserted into the joint compartment.

22. An implant according to claim 21 wherein the implant is substantially free of anchoring portions that need to be attached to the bone, cartilage, ligaments or other tissue, yet by its design is capable of being used with minimal translation, rotation, or other undesired movement or dislocation within or from the joint space.

23. An implant according to claim 22 wherein stability of the implant within the joint space is provided by the fixation/congruency of the device to the one or the other of the two joint members.
